(19) United States
(12) Patent Application Publication
(10) Pub. No.: US 2010/0006095 A1
(43) Pub. Date: Jan. 14, 2010

(54) APPARATUS AND METHODS FOR PROVIDING DRY POWDER MEDICAMENT FOR INHALATION

(75) Inventors: Derek Alan Woodcock, Hertfordshire (GB); Chritopher Pynn, Hampshire (GB); James Thompson, Old Amersham (GB)

Correspondence Address: QUARLES & BRADY LLP, 411 E. WISCONSIN AVENUE, SUITE 2040, MILWAUKEE, WI, 53202-4497, US

(73) Assignee: BRITANNIA PHARMACEUTICALS LIMITED, Surrey (GB)

(21) Appl. No.: 11/990,954

(22) PCT Filed: Jan. 16, 2006
(86) PCT No.: PCT/GB2006/000141

§ 371 Date: Jul. 7, 2009

(30) Foreign Application Priority Data
Jan. 14, 2005 (GB) 0500677.0

(51) Int. Cl. A61M 15/00

(52) U.S. Cl. 128/203.15

(57) ABSTRACT

The application describes apparatus and methods for delivering a powdered medicament for inhalation. The medicament is put in a receptacle (100) on a vibration generator (80) operated by fluid pressure, e.g. by means of an eccentric rotor. A flow of gas from a supply (20) is passed into the receptacle (100), producing in concert with the vibration an aerosol which passes out of the receptacle and along a tube (110) to be inhaled by the patient. Both the gas flow supply and vibration may be subject to the operation of a control unit (60), which may time the production of the aerosol according to a patient's breathing cycle, e.g. as part of a ventilator system.

The present invention is concerned with a dry powder inhaler which typically generates an aerosol of powder particles. In particular, the dry powder inhaler may be used for continuous delivery of a medicament to a patient without requiring activation by the patient.

Typically when a patient's medical condition requires continuous administration of a medicament, the equipment generally used to administer the medicament is a nebuliser. Nebulisers work by generating a fine aerosol of liquid particles from a solution of a medicament. This aerosol may be administered to a patient via a mouthpiece, facemask or via an endotracheal tube for a ventilator.

The problem with a nebuliser is that it is not suitable for administering a medicament which cannot be formulated in liquid form. In addition the efficacy of nebulisers is reduced when included in ventilator circuits as the endotracheal tube acts in part as a block to aerosol deposition. Such a system generally comprises a respirable air generator connected to an endotracheal tube. A nebuliser is used with such a system by connecting it to the endotracheal tube. There is the problem that some of the aerosolised medicament is not inhaled by the patient. This is because where there are bends in the endotracheal tube, at least some of the aerosolised medicament impacts the tube, forming a liquid deposit. The use of a nebuliser in conjunction with a ventilator will frequently require disrupting the ventilator circuit in order to insert the nebuliser. While this is standard practice in many units, it is not entirely without risk. The only alternative to this is to take a patient off an automated ventilator whilst a medicament is administered but this is clearly dangerous for the patient.

To administer a powdered medicament, a dry powder inhaler (DPI) is typically used. In a DPI device, no propellant is used but instead the device relies upon a burst of inspired air drawn through the unit by the patient to aerosolise the medicament. These devices suffer from the problem that they require activation by the patient and are unable to supply an aerosolised medicament continuously.

A way of ameliorating these problems has been sought.

In one aspect the present invention provides a delivery system for a powdered medicament comprising:
a vibration generator, preferably powered by a pressurised fluid supply;
a gas supply; and
a receptacle for a powdered medicament which receptacle has an inlet connected to the gas supply;
   wherein the receptacle is connected to, e.g. supported by, the vibration generator, for the vibration and the supply of gas to the receptacle to cause a powder in the receptacle to fluidise in use, generating an aerosol.

One advantage of the present invention is that the invention provides a simple delivery system for a powdered medicament which can be used to provide a continuous supply of a medicament. Thus the system according to the invention provides a way of delivering a powdered medicament in a similar manner to how a nebuliser delivers a liquid medicament.

A further advantage of the invention is that the aerosol is generated by fluid pressure. Thus in a hospital environment where pressurised cylinders of gases such as oxygen or air are commonly available, the system is easy to operate at a minimal expense.

A further advantage of the present invention is that it is suitable for administering cohesive powders. Pumactant which is a blend of dipalmitoylphosphatidylcholine (DPPC) and phosphatidylglycerol (PG) (DPPC:PG 7:3), is very cohesive due to its low particle size, high moisture affinity and predominantly amorphous structure. It has surprisingly been found that the present invention is suitable for administering a respirable dose of pumactant.

Any known vibration generator, e.g. pneumatic, may be used in the present invention. For ease of use, the vibration generator is preferably compact. Preferably the vibration generator comprises an asymmetrically weighted (eccentric) rotor. Such a rotor may be driven by piston or turbine, preferably turbine.

The system preferably comprises a control unit for controlling the gas supply such that the gas supply is an even supply of gas without fluctuation in the flow rate or pressure of the gas. The control unit is preferably adapted to generate a pulsed gas supply, e.g. so that the flow of gas is intermittent. The control unit is preferably further adapted to monitor the pressure output and duration of the pulsed gas supply.

The control unit may also control the vibration generator in order that the generation of vibration may be timed to be at the same time or shortly before the flow of gas from the control unit.

The gas used in the gas supply is preferably carbon dioxide, nitrogen, air, or a halocarbon (e.g. a fluorocarbon such as HFA-134a or HFC-227); more preferably the gas is carbon dioxide. Where the powdered medicament is hygroscopic or otherwise sensitive to water, the gas supply is preferably a supply of dry gas. Alternatively, the gas supply preferably passes a dryer, such as exposure to a body of a desiccant material, to dry the gas before it reaches the receptacle. Any known desiccants used to dry a supply of gas may be used, such as activated alumina, a silica gel or a molecular sieve.

The receptacle has an outlet from which the aerosol is discharged. The outlet is preferably connected to an outlet tube. The outlet tube may be provided with a connector means suitable for linking the gas outlet with a ventilator or other assisted breathing system used in a hospital environment. Alternatively, the outlet tube may be provided in the form of an endotracheal tube. As a further alternative, the outlet tube may be provided with a mask to aid a patient to inhale the aerosol. The outlet tube generally has a proximal end and a distal end. The proximal end of the outlet tube is connected to the outlet of the receptacle. The distal end of the outlet tube is preferably provided with a one way valve to prevent contamination of the receptacle, e.g. between pulses of a pulsed gas supply.

Where the system of the invention is used in association with a ventilator, the control unit may be functionally linked to the ventilator, e.g. electronically by an electric cable or wirelessly. This is in order that the control unit can time the supply of gas such that the aerosol produced by the system of the invention at the distal end of the outlet tube coincides with the respirable air generated at the corresponding point of the endotracheal tube of the ventilator such that the patient inhales the aerosol into his or her lungs. Optionally the control unit may also control the vibration generator such that the vibration of the receptacle is also timed to coincide with the inspiratory cycle of the ventilator.

According to a further aspect of the present invention there is provided a method of dispensing a powdered medicament to a patient in need of such treatment which method comprises the steps of:
(a) providing a receptacle containing the powdered medicament;
(b) vibrating the receptacle using a vibration generator;
(c) passing a stream of gas through the receptacle to generate an aerosol of the powdered medicament;
   wherein the vibration generator is preferably powered by a pressurised fluid supply as above.

The steps of this method may be performed sequentially, or steps (b) and (c) may be carried out simultaneously.

The method preferably includes a further step (d) of discharging the aerosol preferably through an outlet tube as described above.

Step (c) preferably comprises passing a controlled pulse of gas through the receptacle to generate an aerosol of the powdered medicament. The pulse of gas preferably has controlled duration and pressure. It is preferably a pulse of gas having an even flow.

Where the patient is using an assisted breathing system which generates respirable air for the patient, step (c) preferably comprises producing a series of pulses of gas such that the aerosol is generated and supplied through an outlet at substantially the same time as or in synchronicity with the generation of respirable air by the assisted breathing system.

The receptacle containing the medicament can be any suitable packaging container, for example, a glass or plastic vial or a blister pack. Typically the opening of the receptacle is sealed to preserve sterility of the powder and/or to avoid water adsorption.

The receptacle may contain a single dose of powder for one-time use, or sufficient powder for several doses. It preferably contains from 1 to 20 doses, each containing from 1 to 20 mg of powder; e.g. 10 or 20 doses of 10 mg. or 10 or 20 doses of 2 mg. The medicament is preferably in the form of a respirable powder.

Preferably the medicament is in the form of a respirable powder. Preferably the medicament is in the form of powder particles having a mass median aerodynamic diameter (MMAD) measured by laser diffraction of less than 50 μm, preferably less than 20 μm, more preferably less than 10 μm, most preferably less than 5 μm, particularly preferably from 1 μm to 5 μm.

The system of the invention can be used to administer any medicament suitable for administration by inhalation such as a SAPL (surface active phospholipid) composition, such as pumactant; a bronchodilator (a β₂-agonist or an anti-cholinergic agent), a steroid (such as budesonide), a mucolytic agent, an enzyme (e.g. α1-trypsin), a chemotherapy agent, an immune suppressant (e.g. cyclosporine), a systemic treatment (such as insulin).

The invention is illustrated by way of example by the Figures of the accompanying drawings in which:

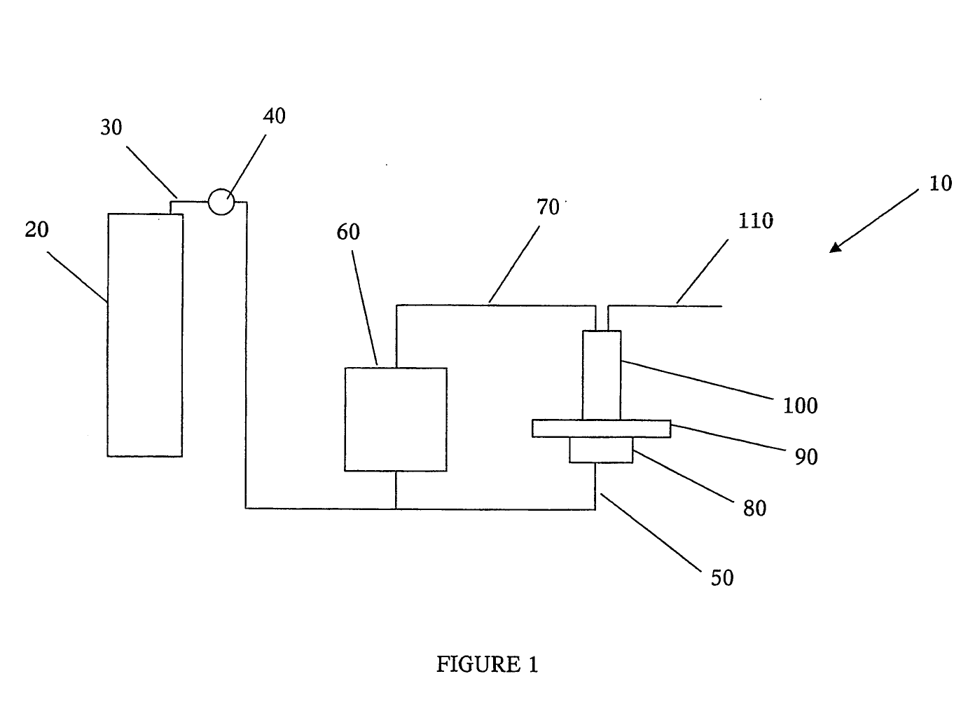

FIG. 1 is a schematic view of a system according to the invention;

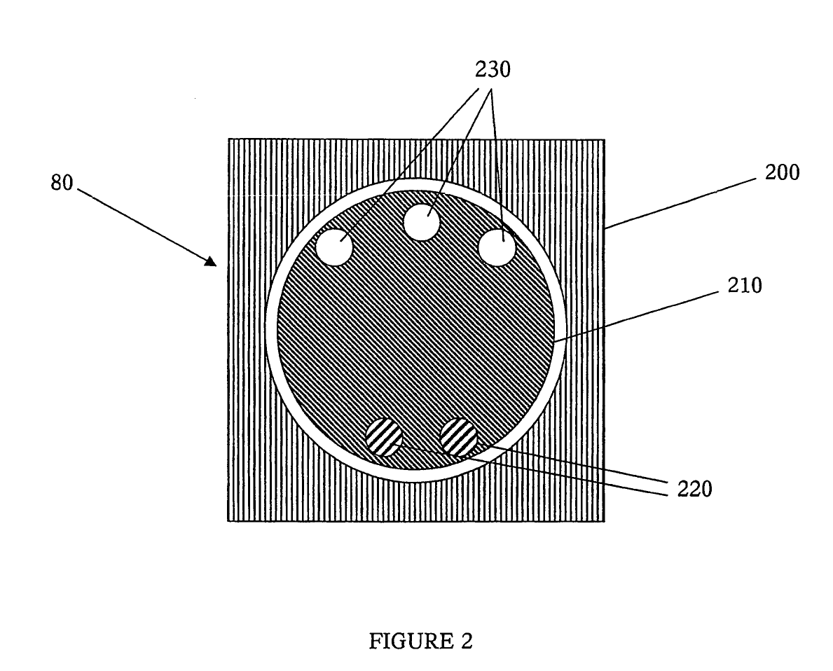

FIG. 2 is a cross-sectional view of a vibration generator suitable for use in a system according to the invention;

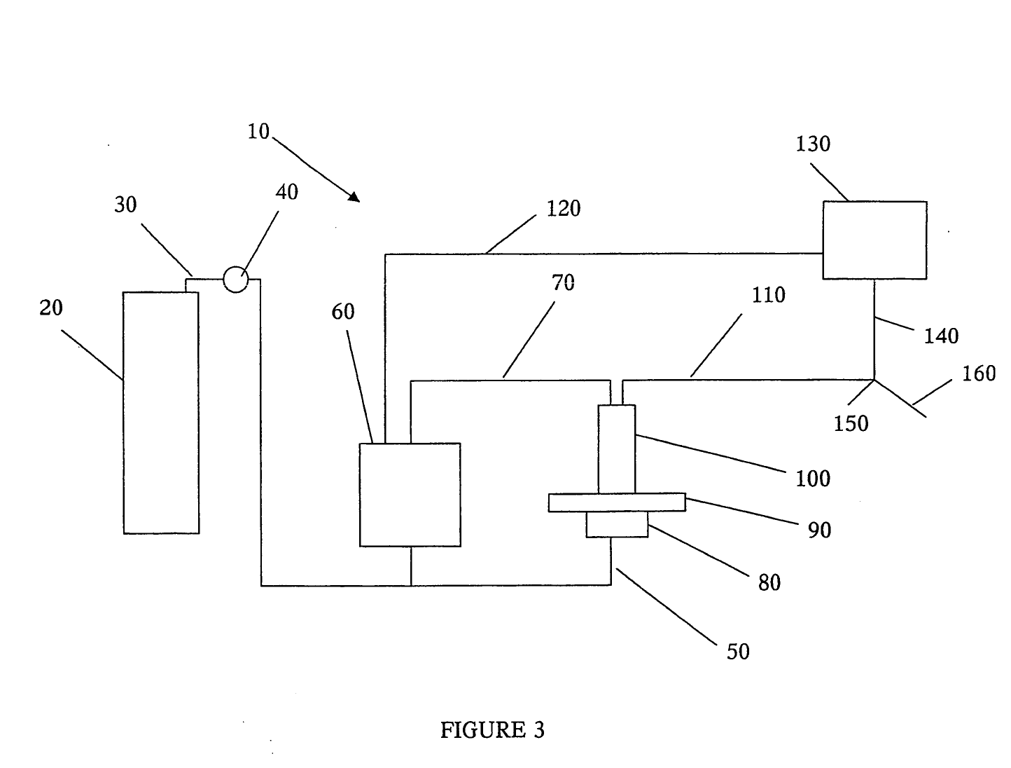

FIG. 3 is a schematic view of a system according to the invention connected to a ventilator unit; and

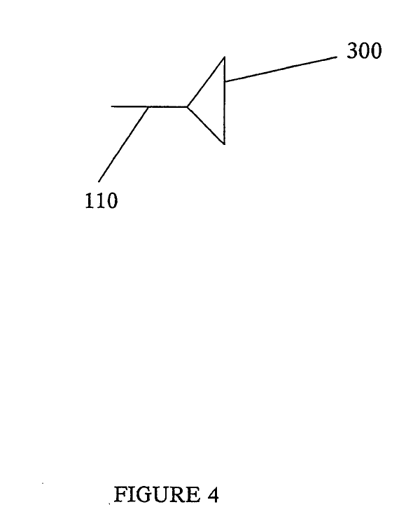

FIG. 4 shows a schematic view of an output tube fitting.

FIG. 1 shows a system 10 embodying the invention having a pressurised gas cylinder 20 containing carbon dioxide, a gas supply line 30 leading from the cylinder 20 to a gas pressure regulator 40. The gas pressure regulator 40 is connected to a control unit 60 and a vibration generator 80 by gas supply lines 50.

A table 90 is supported by the vibration generator 80. A receptacle 100 is placed on the table 90. The receptacle has a gas inlet line 70 which is connected to the control unit 60 and a gas outlet line 110. The receptacle contains the powder to be aerosolised.

Control unit 60 controls the duration and flow-of pulses of gas from cylinder 20. Control unit 60 comprises a solenoid valve with electronic flow control (not shown). The solenoid valve of the control unit 60 is a high purity, grease-free valve. The output pressure of the control unit is from 2 to 100 kPa. Optionally, the control unit may also control the vibration generator 80 in order that the generation of vibration may be timed to be at the same time or shortly before the flow of gas from the control unit.

As an alternative to the embodiment shown in FIG. 1, the table 90 can be formed by the vibration generator 80 such that the receptacle 100 is placed directly on the vibration generator. Also pressurised gas cylinder 20 could be replaced by two pressurised gas cylinders, one connected to the vibration generator 80 and the other connected to the control unit 60.

Vibration generator 80 is shown in more detail in FIG. 2. It comprises a body 200 generally formed from a metal casting or extrusion; the body 200 has air inlet and outlet channels. Body 200 supports a turbine wheel 210 which has paddles (not shown) such that in use the turbine wheel 210 is caused to rotate by the application of gas pressure from the cylinder 20.

The turbine wheel 210 has high density sections 220 forming positive elements. The turbine wheel 210 forms cavities 230 which are negative elements. Rotation of the turbine wheel 210 causes the table 90 and receptacle 100 to vibrate by the centrifugal force of the positive and negative unbalance elements in the turbine wheel 210. The turbine wheel is supported on two pre-lubricated matched sealed bearings (not shown).

In use, the gas cylinder 20 and regulator 40 are adjusted to supply a gas pressure of about 3 Bar. This activates vibration generator 80 which causes a powder (not shown) in the receptacle to fluidise by vibrating the receptacle at a rate of about 12,000 vibrations per minute. Control unit 60 is then activated to supply a flow of gas to the receptacle 100 at a pressure of about 1 Bar through gas inlet line 70 so that an aerosol of fluidised powder is emitted from the receptacle 100 through gas outlet line 110.

In FIG. 3, the system 10 of the invention is shown in use, connected to a ventilator 130. Like numerals are used in FIG. 3 to describe like features in the earlier Figures. In particular, the control unit 60 is in electronic communication with the ventilator 130 which is indicated by 120. The electronic communication is shown in the form of an electric cable but as an alternative may be wireless electronic communication. The ventilator generates respirable air through endotracheal tube 140,160. The outlet tube 110 is connected to endotracheal tube 140 at connector 150. The distal end of the outlet tube 110 at the connector 150 is provided with a one way valve (not shown). This is because the respirable air generated by the ventilator is usually humid respirable air. The one way valve prevents flow of the respirable air into the receptacle 100.

The control unit 60 is in electronic communication with the ventilator in order that the flow of gas from the control unit can be timed such that the outlet tube 110 provides an aerosol of powdered medicament at connector 150 at a time to coincide with the production of respirable air by the ventilator.

As an alternative to the embodiment shown in FIG. 3, the outlet tube 110 does not connect to the endotracheal tube 140,160 but is instead in the form of an endotracheal tube itself. The outlet endotracheal tube 110 is then arranged in parallel with the ventilator endotracheal tube 140,160. In this arrangement, the ventilator and control unit are arranged to generate respirable air and an aerosol, respectively, at the distal ends of the endotracheal tubes 140,160 and 110 at about the same time. This is to ensure that the aerosol is produced when the patient is inhaling.

FIG. 4 shows an alternative embodiment to FIG. 3 where the outlet tube 110 of the system 10 is connected to a inhalation mask 300 which is suitable for a patient to wear on their face to cover their mouth and nose such that they can inhale the aerosol generated by the system 10.

1. A delivery system for a powdered medicament comprising:
a vibration generator which is powered by a pressurised fluid supply;
a gas supply; and
a receptacle for a powdered medicament which receptacle has an inlet connected to the gas supply;
wherein the receptacle is connected to the vibration generator such that the vibration and the supply of gas to the receptacle cause the powder in the receptacle to fluidise generating an aerosol.

2. A system according to claim 1 comprising a control unit programmed to control the gas supply to generate a timed series of gas flow pulses.

3. A system according to claim 1 wherein the vibration generator comprises an asymmetrically weighted rotor.

4. A system according to claim 1 in which a control unit is operable to control the gas supply to give an even supply of gas, without fluctuation in flow rate or pressure of the gas when flowing.

5. A system according to claim 1 wherein a control unit is operable to control the vibration generator in order that the generation of vibrations is timed to be inregister with e.g. at the same time as or shortly before, a period of gas supply from the control unit.

6. A system according to claim 1 wherein a control unit is further adapted to monitor the pressure output and duration of the gas supply.

7. A system according to claim 1 wherein the gas used in the gas supply is carbon dioxide, nitrogen, air, or a halocarbon.

8. A system according to claim 1 wherein the gas supply is a supply of dry gas.

9. A system according to claim 1 wherein the receptacle has an outlet from which the aerosol is discharged which outlet is connected to an outlet tube.

10. A system according to claim 9 wherein the outlet tube is provided with a connector suitable for linking the gas outlet with an assisted breathing system or with an inhalation mask.

11. A system according to claim 9 wherein the outlet tube is in the form of an endotracheal tube.

12. A system according to claim 9 wherein the distal end of the outlet tube is provided with a one way valve to prevent contamination of the receptacle.

13. A system according to claim 10 wherein the system comprises a control unit as defined in claim 3 wherein the control unit is adapted to communicate with an assisted breathing system such that the discharge of the aerosol can be timed by the control system to coincide with production of respirable air by the assisted breathing system.

14. A system according to claim 1 wherein the medicament is in the form of a respirable powder.

15. A system according to claim 1 wherein the receptacle contains from 1 to 20 doses, each containing from 1 to 20 mg of powder.

16. A method of dispensing a powdered medicament to a patient in need of such treatment which method comprises the steps of:
(a) providing a receptacle containing the powdered medicament;
(b) vibrating the receptacle using a vibration generator;
(c) passing a stream of gas through the receptacle to generate an aerosol of the powdered medicament;
wherein the vibration generator is powered by a pressurised fluid supply.

17. A method according to claim 16 wherein the steps of the method of the invention are performed sequentially.

18. A method according to claim 16 wherein steps (b) and (c) are carried out simultaneously after step (a).

19. A method according to claim 16 which includes a further step (d) of discharging the aerosol.

20. A method according to claim 16 wherein step (c) comprises passing a controlled pulse of gas through the receptacle to generate an aerosol of the powdered medicament.

21. A method according to claim 20 wherein the patient is using an assisted breathing system which generates respirable air for the patient and wherein in step (c) the pulse of gas is controlled such that the aerosol is generated at substantially the same time as the assisted breathing system generates the respirable air.

22. A method according to claim 16 wherein the medicament is in the form of a respirable powder.

23. A method according to claim 16 wherein the gas is carbon dioxide, nitrogen, air, or a halocarbon.

24. A method according to claim 16 wherein the stream of gas is a dry stream of gas.

25. A system according to claim 1 wherein the gas used in the gas supply is carbon dioxide.

26. A system according to claim 1 wherein the medicament is in the form of a powder having a mass median aerodynamic diameter (MMAD) measured by laser diffraction of less than 50 μm.

27. A method according to claim 19 wherein in step (d) the aerosol is discharged through an outlet tube as claimed in claim 9.

28. A method according to claim 20 wherein the controlled pulse of gas has controlled duration and pressure.

29. A method according to claim 22 wherein the medicament is in the form of powder particles having a mass median aerodynamic diameter (MMAD) measured by laser diffraction of less than 50 μm.

30. A method according to claim 23 wherein the gas is carbon dioxide.
